(19) United States
(12) Patent Application Publication
(10) Pub. No.: US 2003/0216460 A1
(43) Pub. Date: Nov. 20, 2003

(54) N3 alkylated benzimidazole derivatives as MEK inhibitors

(76) Inventors: Eli M. Wallace, Lyons, CO (US); Joseph P. Lyssikatos, Superior, CO (US); Allison L. Marlow, Boulder, CO (US); T. Brian Hurley, Boulder, CO (US)

Correspondence Address: Stephen H. Docter, McDonnell Boehnen Hulbert & Berghoff, 32nd Floor, 300 S. Wacker Drive, Chicago, IL, 60606, US

(21) Appl. No.: 10/387,682

(22) Filed: Mar. 13, 2003

Related U.S. Application Data
(60) Provisional application No. 60/364,164, filed on Mar. 13, 2002.

(51) Int. Cl. A61K 31/4184; A61K 31/655; C07D 235/08

(52) U.S. Cl. 514/394; 514/151; 548/304.4

(57) ABSTRACT

Disclosed are compounds of the formula I

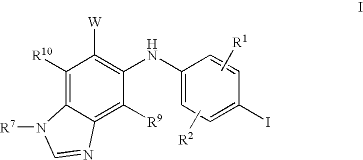

and pharmaceutically acceptable salts and prodrugs thereof,

wherein W, t, R¹, R², R⁷, R⁹, R¹⁰, R¹¹ and R¹² are as defined in the specification.

Such compounds are MEK inhibitors and useful in the treatment of hyperproliferative diseases, such as cancer and inflammation, in mammals. Also disclosed is a method of using such compounds in the treatment of hyperproliferative diseases in mammals, and pharmaceutical compositions containing such compounds.

### BACKGROUND OF THE INVENTION

1. Field of the Invention

This invention relates to a series of alkylated (1H-Benzoimidazol-5-yl)-(4-iodo-phenyl)-amine derivatives that are useful in the treatment of hyperproliferative diseases, such as cancer and inflammation, in mammals. This invention also relates to a method of using such compounds in the treatment of hyperproliferative diseases in mammals, especially humans, and to pharmaceutical compositions containing such compounds.

2. Summary of the Related Art

Cell signaling through growth factor receptors and protein kinases is an important regulator of cell growth, proliferation and differentiation. In normal cell growth, growth factors, through receptor activation (i.e. PDGF or EGF and others), activate MAP kinase pathways. One of the most important and most well understood MAP kinase pathways involved in normal and uncontrolled cell growth is the Ras/Raf kinase pathway. Active GTP-bound Ras results in the activation and indirect phosphorylation of Raf kinase. Raf then phosphorylates MEK1 and 2 on two serine residues (S218 and S222 for MEK1 and S222 and S226 for MEK2) (Ahn et al., Methods in Enzymology 2001, 332, 417-431). Activated MEK then phosphorylates its only known substrates, the MAP kinases, ERK1 and 2. ERK phosphorylation by MEK occurs on Y204 and T202 for ERK1 and Y185 and T183 for ERK2 (Ahn et al., Methods in Enzymology 2001, 332, 417-431). Phosphorylated ERK dimerizes and then translocates to the nucleus where it accumulates (Khokhlatchev et al., Cell 1998, 93, 605-615). In the nucleus, ERK is involved in several important cellular functions, including but not limited to nuclear transport, signal transduction, DNA repair, nucleosome assembly and translocation, and mRNA processing and translation (Ahn et al., Molecular Cell 2000, 6, 1343-1354). Overall, treatment of cells with growth factors leads to the activation of ERK1 and 2 which results in proliferation and, in some cases, differentiation (Lewis et al., Adv. Cancer Res. 1998, 74, 49-139).

In proliferative diseases, genetic mutations and/or overexpression of the growth factor receptors, downstream signaling proteins, or protein kinases involved in the ERK kinase pathway lead to uncontrolled cell proliferation and, eventually, tumor formation. For example, some cancers contain mutations which result in the continuous activation of this pathway due to continuous production of growth factors. Other mutations can lead to defects in the deactivation of the activated GTP-bound Ras complex, again resulting in activation of the MAP kinase pathway. Mutated, oncogenic forms of Ras are found in 50% of colon and >90% pancreatic cancers as well as many others types of cancers (Kohl et al., Science 1993, 260, 1834-1837). Recently, bRaf mutations have been identified in more than 60% of malignant melanoma (Davies, H. et al., Nature 2002, 417, 949-954). These mutations in bRaf result in a constitutively active MAP kinase cascade. Studies of primary tumor samples and cell lines have also shown constitutive or overactivation of the MAP kinase pathway in cancers of pancreas, colon, lung, ovary and kidney (Hoshino, R. et al., Oncogene 1999, 18, 813-822). Hence, there is a strong correlation between cancers and an overactive MAP kinase pathway resulting from genetic mutations.

As constitutive or overactivation of MAP kinase cascade plays a pivotal role in cell proliferation and differentiation, inhibition of this pathway is believed to be beneficial in hyperproliferative diseases. MEK is a key player in this pathway as it is downstream of Ras and Raf. Additionally, it is an attractive therapeutic target because the only known substrates for MEK phosphorylation are the MAP kinases, ERK1 and 2. Inhibition of MEK has been shown to have potential therapeutic benefit in several studies. For example, small molecule MEK inhibitors have been shown to inhibit human tumor growth in nude mouse xenografts, (Sebolt-Leopold et al., Nature-Medicine 1999, 5 (7), 810-816; Trachet et al., AACR Apr. 6-10, 2002, Poster #5426; Tecle, H. IBC 2^{nd} International Conference of Protein Kinases, Sep. 9-10, 2002), block static allodynia in animals (WO 01/05390 published Jan. 25, 2001) and inhibit growth of acute myeloid leukemia cells (Milella et al J Clin Invest 2001, 108 (6), 851-859).

Small molecule inhibitors of MEK have been disclosed. At least thirteen patent applications have appeared in the last several years: U.S. Pat. No. 5,525,625 filed Jan. 24, 1995; WO 98/43960 published Oct. 8, 1998; WO 99/01421 published Jan. 14, 1999; WO 99/01426 published Jan. 14, 1999; WO 00/41505 published Jul. 20, 2000; WO 00/42002 published Jul. 20, 2000; WO 00/42003 published Jul. 20, 2000; WO 00/41994 published Jul. 20, 2000; WO 00/42022 published Jul. 20, 2000; WO 00/42029 published Jul. 20, 2000; WO 00/68201 published Nov. 16, 2000; WO 01/68619 published Sep. 20, 2001; and WO 02/06213 published Jan. 24, 2002.

### SUMMARY OF THE INVENTION

This invention provides for alkylated (1H-Benzoimidazol-5-yl)-(4-iodo-phenyl)-amine compounds of formula I, and pharmaceutically acceptable salts and prodrugs thereof, that are useful in the treatment of hyperproliferative diseases. Specifically, the present invention relates to compounds of formula I that act as MEK inhibitors. Also provided are formulations containing compounds of formula I and methods of using the compounds to treat a patient in need thereof. In addition, there are described processes for preparing the inhibitory compounds of formula I.

Accordingly, the present invention refers to compounds of the formula I:

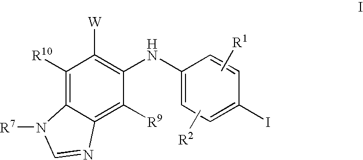

and pharmaceutically accepted salts, prodrugs and solvates thereof, wherein:

R¹, R², R⁹ and R¹⁰ are independently selected from hydrogen, halogen, cyano, nitro, trifluoromethyl, difluoromethoxy, trifluoromethoxy, azido, —OR³, —C(O)R³, —C(O)OR³, NR⁴C(O)OR⁶, —OC(O)R³, —NR⁴SO₂R⁶, —SO₂NR³R⁴, —NR⁴C(O)R³, —C(O)NR³R⁴, —NR⁵C(O)NR³R⁴, —NR⁵C(NCN)NR³R⁴, —NR³R⁴, and

C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR⁴R⁵)ₘ-aryl, aryl, arylalkyl, heteroaryl, heteroarylalkyl, heterocyclyl, heterocyclylalkyl, —O(CR⁴R⁵)ₘ-aryl, —NR⁴(CR⁴R⁵)ₘ-aryl, —O(CR⁴R⁵)ₘ-heteroaryl, —NR⁴(CR⁴R⁵)ₘ-heteroaryl, —O(CR⁴R⁵)ₘ-heterocyclyl and —NR⁴(CR⁴R⁵)ₘ-heterocyclyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —NR⁴C(O)OR, —NR⁴C(O)R³, —C(O)NR³R⁴, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl;

R³ is selected from hydrogen, trifluoromethyl, and

C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR SO₂R″″, —SO₂NR′R″, —C(O)R′, —C(O)OR′, —OC(O)R′, —NR′C(O)OR″″, NR′C(O)R″, —C(O)NR′R″, —SR″″, —S(O)R″″, —SO₂R′, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl;

R′, R″ and R′″ independently are selected from hydrogen, lower alkyl, lower alkenyl, aryl and arylalkyl;

R″″ is selected from lower alkyl, lower alkenyl, aryl and arylalkyl; or

Any two of R′, R″, R′″ or R″″ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl; or

R³ and R⁴ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl; or

R⁴ and R⁵ independently represent hydrogen or C₁-C₆ alkyl; or

R⁴ and R⁵ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR′C(O)OR″,—NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl;

R⁶ is selected from trifluoromethyl; and

C₁-C₁₀ alkyl, C₃-C₁₀ cycloalkyl, aryl, arylalkyl, heteroaryl, heteroarylalkyl, heterocyclyl, and heterocyclylalkyl, where each alkyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR′SO₂R″″, —SO₂NR′R″, —C(O)′R, —C(O)OR′, —OC(O)R′, —NR′C(O)OR″″, —NR′C(O)R″, —C(O)NR′R″, —SO₂R″″, —NR′R′, —NR′C(O)NR″R′″, —NR′C(NCN)NR″R′″, —OR′, aryl, heteroaryl, arylalkyl, heteroarylalkyl, heterocyclyl, and heterocyclylalkyl;

R⁷ is selected from hydrogen; and

C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —NR⁴C(O)OR⁶, —NR⁴C(O)R³, —C(O)NR³R⁴, SO₂R⁶, NR³R⁴, —NR⁵C(O)NR³R⁴, —NR⁵C(NCN)NR³R⁴, OR³, aryl, heteroaryl, arylalkyl, heteroarylalkyl, heterocyclyl, and heterocyclylalkyl;

W is selected from heteroaryl, heterocyclyl, —C(O)OR³, —C(O)NR³R⁴, —C(O)NR⁴OR³, —C(O)R⁴OR³, —C(O)(C₃-C₁₀ cycloalkyl), —C(O)(C₁-C₁₀ alkyl), —C(O)(aryl), —C(O)(heteroaryl) and —C(O)(heterocyclyl), each of which is optionally substituted with 1-5 groups independently selected from

—NR³R⁴, —OR³, —R², and

C₁-C₁₀ alkyl, C₂-C₁₀ alkenyl, and C₂-C₁₀ alkynyl, each of which is optionally substituted with 1 or 2 groups independently selected from —NR³R⁴ and —OR³;

m is 0, 1, 2, 3, 4 or 5; and

j is 1 or 2.

### DETAILED DESCRIPTION OF THE INVENTION

The novel compounds encompassed by the instant invention are those described by the general formula I set forth above, and the pharmaceutically acceptable salts and prodrugs thereof.

The present invention also provides compounds of formula I in which R⁷ is C₁-C₁₀ alkyl, C₃-C₇ cycloalkyl or C₃-C₇ cycloalkylalkyl, each of which can be optionally substituted with 1-3 groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —SO₂R³, —NR⁴C(O)OR⁶, —NR⁴C(O)R³, —C(O)NR³R⁴, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl.

The present invention also provides compounds of formula I wherein R⁹ is hydrogen or halogen, and R¹⁰ is hydrogen.

The present invention also provides compounds of formula I wherein W is —C(O)OR³ or —C(O)NR⁴OR³.

The present invention also provides compounds of formula II:

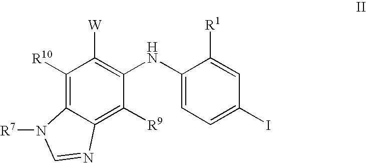

wherein W, R¹, R⁷, R⁹ and R¹⁰ are as defined above for formula I.

The present invention also provides compounds of formula II in which R⁷ is C₁-C₁₀ alkyl, C₃-C₇ cycloalkyl or C₃-C₇ cycloalkylalkyl, each of which can be optionally substituted with 1-3 groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —SO₂R³, —NR⁴C(O)OR⁶, —NR⁴C(O)R³, —C(O)NR³R⁴, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl.

The present invention also provides compounds of formula II wherein R⁹ is hydrogen or halogen, and R¹⁰ is hydrogen.

The present invention also provides compounds of formula II wherein W is —C(O)OR³ or —C(O)NR⁴OR.

The present invention also provides compounds of formula III:

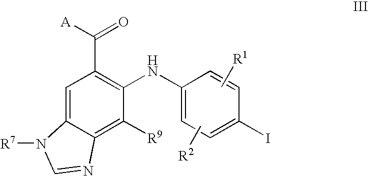

wherein R¹, R², R⁷ and R⁹ are as defined above for formula I, and A is —OR³ or —NR⁴C(O)R³, wherein R³ and R⁴ are as defined above for formula I.

The present invention also provides compounds of formula III in which R⁷ is C₁-C₁₀ alkyl, C₃-C₇ cycloalkyl or C₃-C₇ cycloalkylalkyl, each of which can be optionally substituted with 1-3 groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —SO₂R³, —NR⁴C(O)OR⁶, —NR⁴C(O)R³, —C(O)NR³R⁴—NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl.

The present invention also provides compounds of formula III wherein R⁹ is hydrogen or halogen.

The present invention also provides compounds of formula III wherein R³ is hydrogen or lower alkyl when A is —OR³; and R⁴ is hydrogen when A is —NR⁴C(O)R³.

The present invention also provides compounds of formula IIIa:

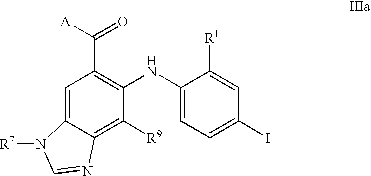

wherein R¹, R², R⁷ and R⁹ are as defined above for formula I, and A is —OR³ or —NR⁴C(O)R³, wherein R³ and R⁴ are as defined above for formula I.

The present invention also provides compounds of formula IIIa in which R⁷ is C₁-C₁₀alkyl, C₃-C₇ cycloalkyl or C₃-C₇ cycloalkylalkyl, each of which can be optionally substituted with 1-3 groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —SO₂R³, —NR⁴C(O)OR⁶, —NR⁴C(O)R³, —C(O)NR³R⁴, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl.

The present invention also provides compounds of formula IIIa wherein R⁹ is hydrogen or halogen.

The present invention also provides compounds of formula IIIa wherein R³ is hydrogen or lower alkyl when A is —OR³; and R⁴ is hydrogen when A is —NR⁴C(O)R³.

Except as expressly defined otherwise, the following definition of terms is employed throughout this specification.

By “C₁-C₁₀ alkyl”, “alkyl” and “lower alkyl” in the present invention is meant straight or branched chain alkyl groups having 1-10 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, heptyl, octyl, and the like. Preferred alkyl radicals are C₁₋₆ alkyl. More preferred alkyl radicals are C₁₋₃ alkyl.

By “C₂-C₁₀ alkenyl”, “lower alkenyl” and “alkenyl” means straight and branched hydrocarbon radicals having from 2 to 10 carbon atoms and at least one double bond and includes ethenyl, propenyl, 1-but-3-enyl, 1-pent-3-enyl, 1-hex-5-enyl and the like. More preferred are lower alkenyl having 3-5 carbon atoms.

By “C₂-C₁₀ alkynyl”, “lower alkynyl” and “alkynyl” means straight and branched hydrocarbon radicals having from 2 to 10 carbon atoms and at least one triple bond and includes ethynyl, propynyl, butynyl, pentyn-2-yl and the like. More preferred are alkynyl having 3-5 carbon atoms.

By the term “halogen” in the present invention is meant fluorine, bromine, chlorine, and iodine.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl), which is optionally mono-, di-, or trisubstituted with, e.g., halogen, lower alkyl, lower alkoxy, trifluoromethyl, aryl, heteroaryl, and hydroxy.

By “heteroaryl” is meant one or more aromatic ring systems of 5-, 6-, or 7-membered rings which includes fused ring systems (at least one of which is aromatic) of 5-10 atoms containing at least one and up to four heteroatoms selected from nitrogen, oxygen, or sulfur. Examples of heteroaryl groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Spiro moieties are also included within the scope of this definition. Heteroaryl groups are optionally mono-, di-, or trisubstituted with, e.g., halogen, lower alkyl, lower alkoxy, haloalkyl, aryl, heteroaryl, and hydroxy.

As used herein, the term “carbocycle”, “carbocyclyl”, “cycloalkyl” or “C₃-C₁₀ cycloalkyl” refers to saturated carbocyclic radicals having three to ten carbon atoms. The cycloalkyl can be monocyclic, or a polycyclic fused system, and can be fused to an aromatic ring. Examples of such radicals include cyclopropyl, cyclobutyl, cyclopentyl and cyclohexyl. The cycloalkyl groups herein are unsubstituted or, as specified, substituted in one or more substitutable positions with various groups. For example, such cycloalkyl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “heterocycle” or “heterocyclyl” is meant one or more carbocyclic ring systems of 5-, 6-, or 7-membered rings which includes fused ring systems of 4-10 atoms containing at least one and up to four heteroatoms selected from nitrogen, oxygen, or sulfur, and with the proviso that the ring of the group does not contain two adjacent O or S atoms. A fused system can be a heterocycle fused to an aromatic group. Preferred heterocycles include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl and quinolizinyl. Spiro moieties are also included within the scope of this definition. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl. The heterocycle groups herein are unsubstituted or, as specified, substituted in one or more substitutable positions with various groups. For example, such heterocycle groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

The term “arylalkyl” means an alkyl moiety (as defined above) substituted with one or more aryl moiety (also as defined above). More preferred arylalkyl radicals are aryl-C₁₋₃-alkyls. Examples include benzyl, phenylethyl, and the like.

The term “heteroarylalkyl” means an alkyl moiety (as defined above) substituted with a heteroaryl moiety (also as defined above). More preferred heteroarylalkyl radicals are 5- or 6-membered heteroaryl-C₁₋₃-alkyls. Examples include, oxazolylmethyl, pyridylethyl and the like.

The term “heterocyclylalkyl” means an alkyl moiety (as defined above) substituted with a heterocyclyl moiety (also defined above). More preferred heterocyclylalkyl radicals are 5- or 6-membered heterocyclyl-C₁₋₃-alkyls. Examples include tetrahydropyranylmethyl.

The term “cycloalkylalkyl” means an alkyl moiety (as defined above) substituted with a cycloalkyl moiety (also defined above). More preferred heterocyclyl radicals are 5- or 6-membered cycloalkyl-C₁₋₃-alkyls. Examples include cyclopropylmethyl.

The term “Me” means methyl, “Et” means ethyl, “Bu” means butyl and “Ac” means acetyl.

The phrase “pharmaceutically acceptable salt(s)”, as used herein, unless otherwise indicated, includes salts of acidic and basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of the present invention are those that form non-toxic acid addition salts, i.e., salts containing pharmaceutically acceptable anions, such as the acetate, benzenesulfonate, benzoate, bicarbonate, bisulfate, bitartrate, borate, bromide, calcium, camsylate, carbonate, chloride, clavulanate, citrate, dihydrochloride, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palimitate, pantothenate, phosphate/diphosphate, polygalacturonate, salicylate, stearate, subacetate, succinate, tannate, tartrate, teoclate, tosylate, triethiodode, and valerate salts. Since a single compound of the present invention may include more than one acidic or basic moieties, the compounds of the present invention may include mono, di or tri-salts in a single compound.

In the case of an acidic moiety in a compound of the present invention, a salt may be formed by treatment of a compound of The present invention with a basic compound, particularly an inorganic base. Preferred inorganic salts are those formed with alkali and alkaline earth metals such as lithium, sodium, potassium, barium and calcium. Preferred organic base salts include, for example, ammonium, dibenzylammonium, benzylammonium, 2-hydroxyethylammonium, bis(2-hydroxyethyl)ammonium, phenylethylbenzylamine, dibenzyl-ethylenediamine, and the like salts. Other salts of acidic moieties may include, for example, those salts formed with procaine, quinine and N-methylglusoamine, plus salts formed with basic amino acids such as glycine, ornithine, histidine, phenylglycine, lysine and arginine. An especially preferred salt is a sodium or potassium salt of a compound of The present invention.

With respect to basic moieties, a salt is formed by the treatment of a compound of The present invention with an acidic compound, particularly an inorganic acid. Preferred inorganic salts of this type may include, for example, the hydrochloric, hydrobromic, hydroiodic, sulfuric, phosphoric or the like salts. Preferred organic salts of this type, may include, for example, salts formed with formic, acetic, succinic, citric, lactic, maleic, fumaric, palmitic, cholic, pamoic, mucic, D-glutamic, D-camphoric, glutaric, glycolic, phthalic, tartaric, lauric, stearic, salicyclic, methanesulfonic, benzenesulfonic, paratoluenesulfonic, sorbic, puric, benzoic, cinnamic and the like organic acids. An especially preferred salt of this type is a hydrochloride or sulfate salt of a compound of The present invention.

In the compounds of the present invention, where terms such as (CR⁴R⁵)ₘ or (CR⁴R⁵)ₜ are used, R⁴ and R⁵ may vary with each iteration of m or t above 1. For instance, where m or t is 2, the terms (CR⁴R⁵)ₘ or (CR⁴R⁵)ₜ may equal —CH₂CH₂— or —CH(CH₃)C(CH₂CH₃)(CH₂CH₂CH₃)— or any number of similar moieties falling within the scope of the definitions of R⁴ and R⁵.

Certain compounds of the present invention may have asymmetric centers and therefore exist in different enantiomeric forms. All optical isomers and stereoisomers of the compounds of the present invention, and mixtures thereof, are considered to be within the scope of the invention. With respect to the compounds of the present invention, the invention includes the use of a racemate, one or more enantiomeric forms, one or more diastereomeric forms, or mixtures thereof. The compounds of the present invention may also exist as tautomers. This invention relates to the use of all such tautomers and mixtures thereof.

The subject invention also includes isotopically-labeled compounds, which are identical to those recited in the present invention, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹p, ³²P, ³⁵S, 18F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Futher, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compound of the present invention and prodrugs thereof can generally be prepared by carrying out procedures disclosed in the Schemes and/or in the Examples and Preparations below, by substituting a readily available isotopically labeled reagent for a non-isotopically labeled reagent.

This invention also encompasses pharmaceutical compositions containing and methods of treating proliferative disorders, or abnormal cell growth, by administering prodrugs of compounds of the the present invention. Compounds of the present invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the present invention. The amino acid residues include but are not limited to the 20 naturally occurring amino acids commonly designated by three letter symbols and also includes 4-hydroxyproline, hydroxylysine, demosine, isodemosine, 3-methylhistidine, norvaline, beta-alanine, gamma-aminobutyric acid, cirtulline, homocysteine, homoserine, omithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

It is to be understood that in instances where two or more radicals are used in succession to define a substituent attached to a structure, the first named radical is considered to be terminal and the last named radical is considered to be attached to the structure in question. Thus, for example, the radical arylalkyl is attached to the structure in question by the alkyl group.

The invention also relates to a pharmaceutical composition for the treatment of a hyperproliferative disorder in a mammal which comprises a therapeutically effective amount of a compound of the present invention, or a pharmaceutically acceptable salt, prodrug or hydrate thereof, and a pharmaceutically acceptable carrier. In one embodiment, said pharmaceutical composition is for the treatment of cancer such as brain, lung, squamous cell, bladder, gastic, pancreatic, breast, head, neck, renal, kidney, ovarian, prostate, colorectal, oesohageal, testicular, gynecological or thyroid cancer. In another embodiment, said pharmaceutical composition is for the treatment of a non-cancerous hyperproliferative disorder such as benign hyperplasia of the skin (e.g., psoriasis), restenosis, or prostate (e.g., benign prostatic hypertrophy (BPH)).

The invention also relates to a pharmaceutical composition for the treatment of pancreatitis or kidney disease (including proliferative glomerulonephritis and diabetes-induced renal disease) or pain in a mammal which comprises a therapeutically effective amount of a compound of the present invention, or a pharmaceutically acceptable salt, prodrug or hydrate thereof, and a pharmaceutically acceptable carrier.

The invention also relates to a pharmaceutical composition for the prevention of blastocyte implantation in a mammal which comprises a therapeutically effective amount of a compound of the present invention, or a pharmaceutically acceptable salt, prodrug or hydrate thereof, and a pharmaceutically acceptable carrier.

The invention also relates to a pharmaceutical composition for treating a disease related to vasculogenesis or angiogenesis in a mammal which comprises a therapeutically effective amount of a compound of the present invention, or a pharmaceutically acceptable salt, prodrug or hydrate thereof, and a pharmaceutically acceptable carrier. In one embodiment, said pharmaceutical composition is for treating a disease selected from the group consisting of tumor angiogenesis, chronic inflammatory disease such as rheumatoid arthritis, inflammatory bowel disease, atherosclerosis, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

The invention also relates to a method of treating a hyperproliferative disorder in a mammal that comprises administering to said mammal a therapeutically effective amount of a compound of the present invention, or a pharmaceutically acceptable salt, prodrug or hydrate thereof. In one embodiment, said method relates to the treatment of cancer such as brain, lung, squamous cell, bladder, gastic, pancreatic, breast, head, neck, renal, kidney, ovarian, prostate, colorectal, oesohageal, testicular, gynecological or thyroid cancer. In another embodiment, said method relates to the treatment of a non-cancerous hyperproliferative disorder such as benign hyperplasia of the skin (e.g., psoriasis), restenosis, or prostate (e.g., benign prostatic hypertrophy (BPH)).

The invention also relates to a method for the treatment of a hyperproliferative disorder in a mammal that comprises administering to said mammal a therapeutically effective amount of a compound of the present invention, or a pharmaceutically acceptable salt, prodrug or hydrate thereof, in combination with an anti-tumor agent selected from the group consisting of mitotic inhibitors, alkylating agents, anti-metabolites, intercalating antibiotics, growth factor inhibitors, cell cycle inhibitors, enzyme inhibitors, topoisomerase inhibitors, biological response modifiers, anti-hormones, angiogenesis inhibitors, and anti-androgens.

The invention also relates to a method of treating pancreatitis or kidney disease or pain in a mammal that comprises administering to said mammal a therapeutically effective amount of a compound of the present invention, or a pharmaceutically acceptable salt, prodrug or hydrate thereof.

The invention also relates to a method of preventing blastocyte implantation in a mammal that comprises administering to said mammal a therapeutically effective amount of a compound of the present invention, or a pharmaceutically acceptable salt, prodrug or hydrate thereof.

The invention also relates to a method of treating diseases related to vasculogenesis or angiogenesis in a mammal that comprises administering to said mammal a therapeutically effective amount of a compound of the present invention, or a pharmaceutically acceptable salt, prodrug or hydrate thereof. In one embodiment, said method is for treating a disease selected from the group consisting of tumor angiogenesis, chronic inflammatory disease such as rheumatoid arthritis, atherosclerosis, inflammatory bowel disease, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

Patients that can be treated with compounds of the present invention, or pharmaceutically acceptable salts, prodrugs and hydrates of said compounds, according to the methods of this invention include, for example, patients that have been diagnosed as having psoriasis, restenosis, atherosclerosis, BPH, lung cancer, bone cancer, CMML, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, testicular, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphona, spinal axis tumors, brain stem gliomas or pituitary adenomas).

This invention also relates to a pharmaceutical composition for inhibiting abnormal cell growth in a mammal which comprises an amount of a compound of the present invention, or a pharmaceutically acceptable salt or solvate or prodrug thereof, in combination with an amount of a chemotherapeutic, wherein the amounts of the compound, salt, solvate, or prodrug, and of the chemotherapeutic are together effective in inhibiting abnormal cell growth. Many chemotherapeutics are presently known in the art. In one embodiment, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

This invention further relates to a method for inhibiting abnormal cell growth in a mammal or treating a hyperproliferative disorder which method comprises administering to the mammal an amount of a compound of the present invention, or a pharmaceutically acceptable salt or solvate or prodrug thereof, in combination with radiation therapy, wherein the amounts of the compound, salt, solvate, or prodrug, is in combination with the radiation therapy effective in inhibiting abnormal cell growth or treating the hyperproliferative disorder in the mammal. Techniques for administering radiation therapy are known in the art, and these techniques can be used in the combination therapy described herein. The administration of the compound of the invention in this combination therapy can be determined as described herein.

It is believed that the compounds of the present invention can render abnormal cells more sensitive to treatment with radiation for purposes of killing and/or inhibiting the growth of such cells. Accordingly, this invention further relates to a method for sensitizing abnormal cells in a mammal to treatment with radiation which comprises administering to the mammal an amount of a compound of the present invention or pharmaceutically acceptable salt or solvate or prodrug thereof, which amount is effective is sensitizing abnormal cells to treatment with radiation. The amount of the compound, salt, or solvate in this method can be determined according to the means for ascertaining effective amounts of such compounds described herein.

The invention also relates to a method of and to a pharmaceutical composition of inhibiting abnormal cell growth in a mammal which comprises an amount of a compound of the present invention, or a pharmaceutically acceptable salt or solvate thereof, a prodrug thereof, or an isotopically-labeled derivative thereof, and an amount of one or more substances selected from anti-angiogenesis agents, signal transduction inhibitors, and antiproliferative agents.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound of the present invention and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MMP-1, MMP-3, MMP-4, MMP-5, MMP-6, MMP-7, MMP-8, MMP-10, MMP-11, MMP-12, and MMP-13).

Some specific examples of MMP inhibitors useful in the present invention are AG-3340, RO 32-3555, and RS 13-0830.

The terms “abnormal cell growth” and “hyperproliferative disorder” are used interchangeably in this application.

“Abnormal cell growth”, as used herein, unless otherwise indicated, refers to cell growth that is independent of normal regulatory mechanisms (e.g., loss of contact inhibition). This includes, for example, the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; and (5) benign and malignant cells of other proliferative diseases in which aberrant serine/theroine kinase activation occurs.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

Representative compounds of the present invention, which are encompassed by the present invention include, but are not limited to the compounds of the examples and their pharmaceutically acceptable acid or base addition salts or prodrugs thereof.

The examples presented below are intended to illustrate particular embodiments of the invention, and are not intended to limit the scope of the specification or the claims in any way.

An illustration of the preparation of compounds of the present invention is shown in Schemes 1-3.

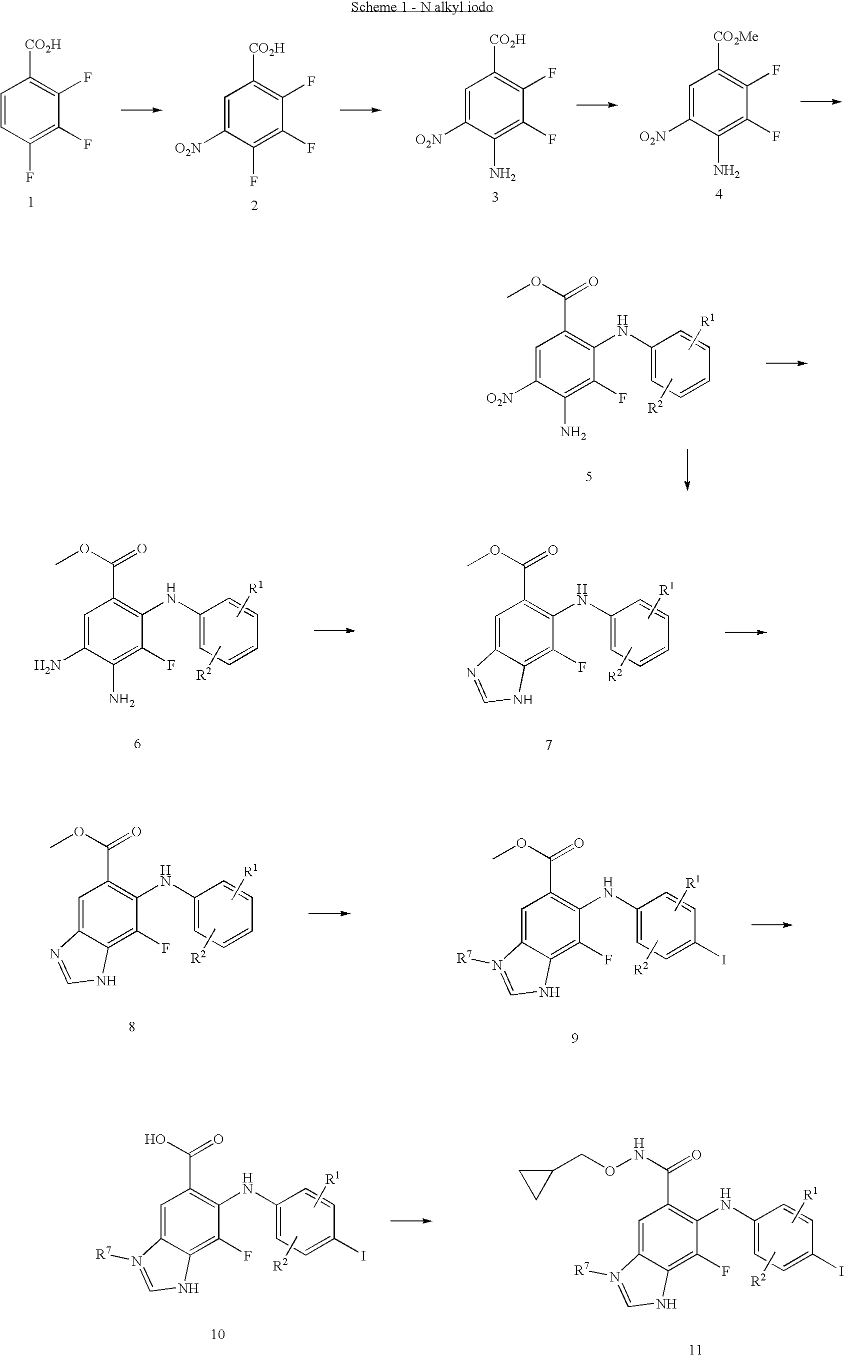

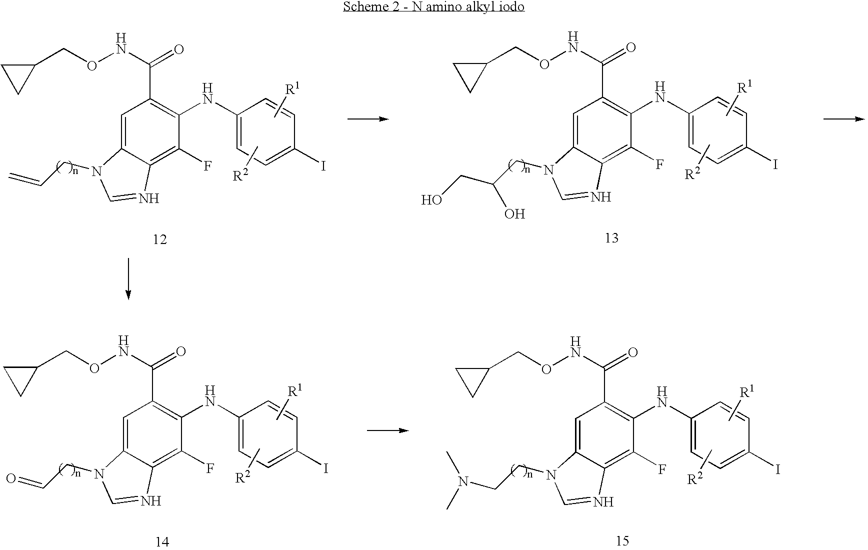

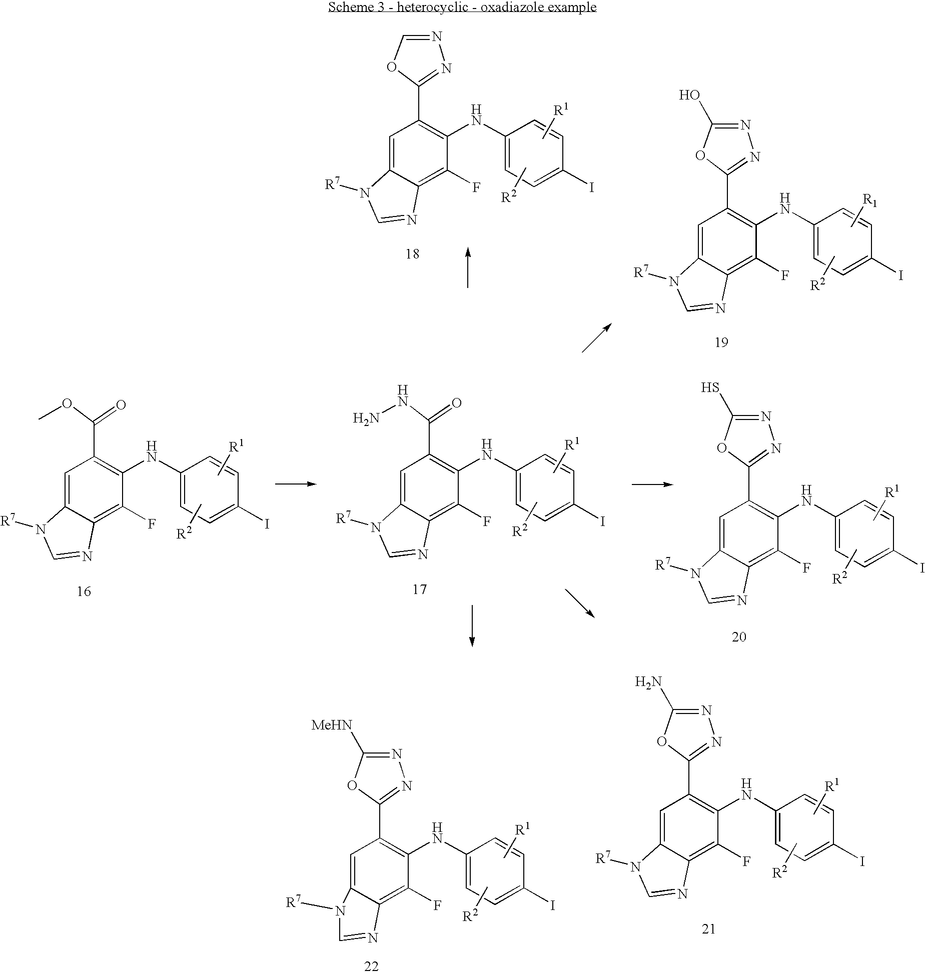

General synthetic methods which may be referred to for preparing some of the compounds of the present invention are provided in PCT published application number WO 00/42022 (published Jul. 20, 2000). The foregoing patent application is incorporated herein by reference in its entirety.

The examples presented below are intended to illustrate particular embodiments of the invention, and are not intended to limit the scope of the specification or the claims in any way.

An illustration of the preparation of compounds of the present invention is shown in Schemes 1-3.

Scheme 1 illustrates the synthesis of compounds of the present invention. In step 1, the acid is nitrated using standard conditions preferable fuming nitric acid in H₂SO₄. In step 2, the aniline is prepared by fluoride displacement with NH₄OH at room temperature in water followed by careful acidification with concentrated mineral acid to pH near 0. In step 3, the ester is prepared by standard methods including by not limited to Fisher Esterification (MeOH, H₂SO₄), and reaction with TMSCHN₂ in suitable organic solvents like PhMe/MeOH or THF/MeOH. In step 4, the dianilino derivative is prepared by heating (60 to 200° C.) the ester with an excess of the appropriate aniline neat or in an organic solvent like xylenes. For example, when R¹=Me and R²=H the preferred method is stirring the ester with 10 equivalents aniline in xylenes at reflux until complete reaction. In step 5, the nitro arene is reduced to produce the diamine by standard reduction conditions, including by not limited to H₂, and Pd/C or Pd(OH)₂/C or Raney Nickel in organic solvent like EtOH or THF, Fe in AcOH, Zn in AcOH or Zn, NH₄Cl (aq) in MeOH. In step 6, the diamine is cyclization by heating with formic acid neat or formamidine acetate in an appropriate solvent like EtOH. Alternatively, when R¹ or R² does not equal halo the nitro arene can be converted directly to the benzimidazole in step 7 by heating in formic acid with Pd(OH)₂/C or other palladium source like Pd/C. In step 8, the iodide is incorporated by standard methods, including but not limited to NIS and pTsOH in organic cosolvents like THF and MeOH or benzyltrimethylammonium dichloroiodinate and ZnCl₂ in AcOH. In step 9, the benzimidazole is alkylated to give a near equal mixture of N1 and N3 products which are separable by standard techniques, including, for example, chromatography and trituration. The alkylation is accomplished by use of an alkylating agent like an alkyl halide and base like NaH, or K₂CO₃ in suitable organic solvent like DMF or THF at temperatures ranging from 0 to 80° C. R⁷ can be further modified by various synthetic methods known in the art, as exemplified below. In step 10, the ester is hydrolysized by standard saponification methods. The acid is then converted to the desired hydroxamate in step 11 by standard coupling procedures including but not limited to EDCI, HOBt or PyBOP and the appropriate hydroxylamine in suitable organic solvents like DMF, THF or methylene chloride.

In Scheme 2, the preparation of N3 alkyl amino benzimidazole derivatives is illustrated. In step 1, the terminal alkene of the N3 alkylated benzimidazole hydroxamate is dihydroxylated using a suitable oxidant like OSO₄ in suitable solvent or KMnO₄ or 12, AgOAc, AcOH, water. The diol is then further oxidized in step 2 by NaIO₄ or Pb(OAc)₄ in suitable biphasic mixture to give the aldehyde. Alternatively (step 3), the alkene can be directly converted to the aldehyde by standard methods including but not limited to ozone/Me₂S, NaIO₄/OsO₄ or KMnO₄. In step 4, the amine is prepared by reductive amination using standard methods such as Na(CN)BH₃, Na(OAC)₃BH, NMe₄BH(OAc)₃ with or without AcOH in a suitable solvent such as methylene chloride, acetonitrile or THF. The preferable reduction amination is to treat the aldehyde with amine, Me4NBH(OAc)₃ and acetic acid in MeCN at room temperature.

Scheme 3 illustrates the preparation of compounds of the present invention where W is heterocyclic. In step 1, the methyl ester is converted to the hydrazide by stirring with hydrazine in a suitable solvent like EtOH at temperatures from 50 to 100° C. The desired heterocyclic derivative is then prepared by cyclization with the appropriate reagent. For oxadiazole 18 the hydrazide is treated with an orthoformate like triethyl orthoformate, and an acid catalyst like pTsOH in a suitable organic solvent like EtOH at elevated temperatures (50-100° C.). For hydroxy oxadiazole 19 the hydrazide can be cyclized with phosgene or a phosgene equivalent like triphosgene or carbonyl diimidazole in a suitable organic solvent like toluene at temperatures ranging from 50 to 120° C. The mercapto oxadizaole 20 can be prepared by reaction with carbon disulfide, and base like KOH in suitable organic solvent like EtOH at elevated temperatures (50-100° C.). The amino oxadiazole 21 can be made by reaction with BrCN and base like NaHCO₃, in a suitable biphasic solvent system like dioxane and water at room temperature. Finally, the substituted amino oxadiazole 22 can be prepared by first reacting the hydrazide with an appropriate isothiocyanate in a suitable organic solvent like DMF or THF at temperatures ranging from 25 to 100° C. The intermediate can be isolated or can be cyclized directly with the treatment of EDCI or other carbodiimide in suitable organic solvent like THF or DMF at temperatures ranging from room temperature to 80° C.

The compounds of the present invention may have asymmetric carbon atoms. Diastereomeric mixtures can be separated into their individual diastereomers on the basis of their physical chemical differences by methods known to those skilled in the art, for example, by chromatography or fractional crystallization. Enantiomers can be separated by converting the enantiomer mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The activity of the compounds of the present invention may be determined by the following procedure. N-terminal 6 His-tagged, constitutively active MEK1 (2-393) is expressed in E. coli and protein is purified by conventional methods (Ahn et al. Science 1994, 265, 966-970). The activity of MEK1 is assessed by measuring the incorporation of γ-³³P-phosphate from γ-³³P-ATP onto N-terminal His tagged ERK2, which is expressed in E. coli and is purified by conventional methods, in the presence of MEK1. The assay is carried out in 96-well polypropylene plate. The incubation mixture (100 μL) comprises of 25 mM Hepes, pH 7.4, 10 mM MgCl₂, 5 mM β-glycerolphosphate, 100 μM Na-orthovanadate, 5 mM DTT, 5 nM MEK1, and 1 μM ERK2. Inhibitors are suspended in DMSO, and all reactions, including controls are performed at a final concentration of 1% DMSO. Reactions are initiated by the addition of 10 μM ATP (with 0.5 μCi γ-³³p-ATP/well) and incubated at ambient temperature for 45 minutes. Equal volume of 25% TCA is added to stop the reaction and precipitate the proteins. Precipitated proteins are trapped onto glass fiber B filterplates, and excess labeled ATP washed off using a Tomtec MACH III harvestor. Plates are allowed to air-dry prior to adding 30 μL/well of Packard Microscint 20, and plates are counted using a Packard TopCount. In this assay, compounds of the invention exhibited an IC₅₀ of less than 50 micromolar.

The following compounds exemplify such activity.

Compound #

11a
11b
11c
11d
11e
11f
11g
11h
11i
11j

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumor substances, for example those selected from, for example, mitotic inhibitors, for example vinblastine; alkylating agents, for example cis-platin, carboplatin and cyclophosphamide; anti-metabolites, for example 5-fluorouracil, cytosine arabinside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example, interferon; and anti-hormones, for example anti-estrogens such as NolvadexTM (tamoxifen) or, for example anti-androgens such as CasodexTM (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Ester, Pa., 15^{th} Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

The disclosures in this application of all articles and references, including patents, are incorporated herein by reference.

The invention is illustrated further by the following examples which are not to be construed as limiting the invention in scope or spirit to the specific procedures described in them.

The starting materials and various intermediates may be obtained from commercial sources, prepared from commercially available organic compounds, or prepared using well known synthetic methods.

Representative examples of methods for preparing intermediates of the invention are set forth below.

### EXAMPLES

### Example 1

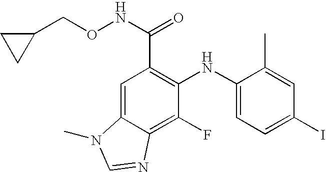

7-Fluoro-6-(4-iodo-2-methyl-phenylamino)-3-methyl-3H-benzoimidazole-5-carboxylic acid cyclopropylmethoxy-amide (11a)

Step A: 2,3,4-Trifluoro-5-nitro-benzoic acid

A 3 liter three neck round bottom flask is charged with 125 ml H₂SO₄. Fuming nitric acid is added (8.4 ml, 199 mmol) and the mixture gently stirred. 2,3,4-Trifluorobenzoic acid (25 g, 142 mmol) is added in 5 g portions over 90 minutes. The dark brownish yellow solution is stirred for 60 min at which time the reaction is complete. The reaction mixture is poured into 1 liter of an ice:water mixture and extracted with diethyl ether (3×600 ml). The combined organic extracts are dried (MgSO₄) and concentrated under reduced pressure to give a yellow solid. The solid is suspended in hexanes and stirred for 30 min after which time it is filtered to give 29 g (92%) of clean desired product as an off-yellow solid.

Step B: 4-Amino-2,3-difluoro-5-nitro-benzoic acid

Ammonium hydroxide solution (˜30% in water) (35 ml, 271 mmol) is added to a solution of 2,3,4-trifluoro-5-nitro-benzoic acid (15 g, 67.8 mmol) in 30 ml water at 0° C. with stirring. Upon completion of ammonium hydroxide addition the reaction mixture is warmed to room temperature with stirring. After 2.5 h, the reaction mixture is cooled to 0° C. and concentrated HCl is carefully added until pH of reaction mixture is near 0. The reaction mixture is diluted with water (30 ml) and extracted with diethyl ether (3×50 ml). The combined organic extracts are dried (MgSO₄) and concentrated under reduced pressure to give 14 g (95%) of pure desired product.

Step C: 4-Amino-2,3-difluoro-5-nitro-benzoic acid methyl ester

A 2 M solution of TMS diazomethane in hexanes (6.88 ml, 13.75 mmol) is added to a suspension of 4-amino-2,3-difluoro-5-nitro-benzoic acid (2.00 g, 9.17 mmol) in 25 ml of 4:1 THF:MeOH at 0° C. under nitrogen atmosphere. Upon completion of addition, reaction mixture is warmed to room temperature. After 0.5 h, excess TMS diazomethane is destroyed by the careful addition of acetic acid. The reaction is then concentrated under reduced pressure and dried in vacuo to give 1.95 g (92%) of pure desired product.

Step D: 4-Amino-3-fluoro-5-nitro-2-o-tolylamino-benzoic acid methyl ester

4-Amino-2,3-difluoro-5-nitro-benzoic acid methyl ester (12.0 g, 51.7 mmol) is suspended in xylenes (60 ml) and ortho-toluidine is added (55.2 ml, 517 mmol). The reaction mixture is heated to reflux with stirring under a nitrogen atmosphere. After 36 h, the reaction mixture is cooled to room temperature, diluted with diethyl ether and washed with 10% aqueous HCl solution. The aqueous washings are extracted with diethyl ether. The combined organic extracts are concentrated under reduced pressure. The residue is dissolved in methylene chloride and filtered through silica gel in a fritted funnel, rinsing with methylene chloride. Three fractions are recovered. The first (2 liter) is nearly clean. The second (1 liter) and third (1 liter) fractions are only partially pure. The first fraction is concentrated under reduced pressure and triturated with diethyl ether to give 11.2 g (68%) of clean desired product as a bright yellow solid.

Step E: 7-Fluoro-6-o-tolylamino-1H-benzoimidazole-5-carboxylic acid methyl ester

4-Amino-3-fluoro-5-nitro-2-o-tolylamino-benzoic acid methyl ester (1.57 g, 4.92 mmol), formic acid (25 ml, 26.5 mmol) and 20% Pd(OH)₂/C (1.57 g, 2.95 mmol) in 25 ml EtOH are heating with stirring to 95° C. After 16 h, the reaction mixture is cooled to room temperature and 0.5 g 20% Pd(OH)₂/C and 10 ml formic acid added. The reaction mixture is heated to 95° C. with stirring. After 16 h, the reaction mixture is cooled to room temperature and filtered through Celite rinsing with EtOH. The filtrate is concentrated under reduced pressure until the desired product precipitates. The desired product is collected by filtration. The filtrate is concentrated again until more desired product precipitates. The product is collected by filtration. Repeated EtOH concentration, product filtration several times. Recovered 1.09 g (74%) pure desired product.

Step F.: 7-Fluoro-6-(4-iodo-2-methyl-phenylamino)-1H-benzoimidazole-5-carboxylic acid methyl ester

7-Fluoro-6-o-tolylamino-1H-benzoimidazole-5-carboxylic acid methyl ester (1.47 g, 4.92 mmol) is suspended in 1:1 THF:MeOH mixture (44 ml) and cooled to −78° C. under a nitrogen atmosphere. A solution of NIS (1.66 g, 7.39 mmol) in THF (2 ml) is added followed by a MeOH (2 ml) solution of TsOH.H₂O (1.87 g, 9.84 mmol). After 30 min, reaction mixture is warmed to 0° C. and 1 ml methylene chloride is added. The reaction is slowly allowed to warm to room temperature with stirring over 16 h. The reaction mixture is quenched by the addition of 10% Na₂S₂O₄ solution. The reaction mixture is diluted with water and ethyl acetate and the layers separated. The aqueous layer is extracted with ethyl acetate. The combined organic extracts are dried (Na₂SO₄) and concentrated under reduced pressure. The recovered solid is triturated with MeOH to give 1.45 g (69%) pure desired product.

Step G: 7-Fluoro-6-(4-iodo-2-methyl-phenylamino)-3-methyl-3H-benzoimidazole-5-carboxylic acid methyl ester

7-Fluoro-6-(4-iodo-2-methyl-phenylamino)-1H-benzoimidazole-5-carboxylic acid methyl ester (100 mg, 0.235 mmol) is suspended in DMF (2.5 ml) and cooled to 0° C. under an atmosphere of nitrogen. NaH (95%) (6 mg, 0.238 mmol) is added. After 10 min, MeI (15 μl, 0.238 mmol) is added. After 45 min, the reaction mixture is warmed to rt. After 1.5 h, the reaction mixture is quenched with water and diluted with ethyl acetate and brine. The layers were separated and the aqueous layer extracted with ethyl acetate. The combined organic extracts are dried (Na₂SO₄) and concentrated under reduced pressure. The crude product mixture is purified by FCC (10:1 methylene chloride:ethyl acetate) to give 36 mg (36%) of the desired methyl N3 product and 43 mg (43%) of methyl Ni product.

Step H: 7-Fluoro-6-(4-iodo-2-methyl-phenylamino)-3-methyl-3H-benzoimidazole-5-carboxylic acid

7-Fluoro-6-(4-iodo-2-methyl-phenylamino)-3-methyl-3H-benzoimidazole-5-carboxylic acid methyl ester (34 mg, 0.077 mmol) is suspended in 1:1 THF:MeOH (2 ml) and 20% NaOH (500 μl) is added. After 16 h, the reaction mixture is cooled to 0° C. and 1 M HCl solution is added dropwise until pH is 1 to 2. The reaction is diluted with ethyl acetate and water and the layers separated. The organic layer is washed with brine, dried (MgSO₄) and concentrated under reduced pressure to give 33 mg (100%) of desired product as a white solid.

Step I. 7-Fluoro-6-(4-iodo-2-methyl-phenylamino)-3-methyl-3H-benzoimidazole-5-carboxylic acid cyclopropylmethoxy-amide

7-Fluoro-6-(4-iodo-2-methyl-phenylamino)-3-methyl-3H-benzoimidazole-5-carboxylic acid (30 mg, 0.071 mmol) is suspended in DMF (1 ml) and HOBt (11 mg, 0.085 mmol) followed by triethyl amine (22 μl, 0.162 mmol) is added. Cyclopropyl methyl hydroxylamine hydrochloride (10 mg, 0.085 mmol) (WO 0042022) is added followed by EDCI (18 mg, 0.092 mmol). After 16 h, the reaction mixture is diluted with ethyl acetate and water and the layers separated. The organic layer is washed with saturated NH₄Cl, brine, saturated NaHCO₃, water and brine. The organic layer is dried (MgSO₄) and concentrated under reduced pressure. The crude reaction mixture is purified by FCC eluted with 20:1 methylene chloride:MeOH to give 21 mg (61%) pure desired product (11a) as a beige solid: MS APCI (+) m/z 495 (M+1) detected; MS APCI (−) m/z 493 (M−1) detected; ¹H NMR (400 MHz, DMSO-d₆) δ 11.62 (s, 1H), 8.38 (s, 1H), 7.69 (s, 1H), 7.57 (s, 1H), 7.43 (d, 1H), 7.25 (dd, 1H), 6.12 (dd, 1H), 3.89 (s, 3H), 3.58 (d, 2H), 2.23 (s, 3H), 1.01 (m, 1H), 0.47 (m, 2H), 0.19 (m, 2H); ¹⁹F NMR (376 MHz, DMSO-d₆) δ −133.71 (s).

### Example 2

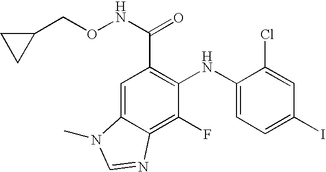

6-(2-Chloro-4-iodo-phenylamino)-7-fluoro-3-methyl-3H-benzoimidazole-5-carboxylic acid cyclopropylmethoxy-amide (11b)

Step A. 4-Amino-3-fluoro-5-nitro-2-phenylamino-benzoic acid methyl ester

4-Amino-2,3-difluoro-5-nitro-benzoic acid methyl ester (23.48 g, 101.1 mmol) is suspended in xylenes (125 mL) and aniline (92 mL, 1011 mmol) is added. The reaction mixture is stirred at 125° C. for 16 hours under N₂. The reaction mixture is cooled to room temperature and solids precipitate out of solution. The solids are collected by filtration and are washed with xylenes and then diethyl ether. Recovered 22.22 g (72.78 mmol) of yellow solid which is pure desired product. The filtrate is concentrated under reduced pressure, redissolved in methylene chloride and flushed through a plug of silica gel eluting with methylene chloride. The desired fractions are concentrated under reduced pressure to give a brown solid which is triturated with diethyl ether to give 5.47 g (17.91 mmol) of yellow solid which is pure desired product. Combined product yield is 27.69 g (90%): MS APCI (−) m/z 304 (M−1) detected.

Step B. 7-Fluoro-6-phenylamino-3H-benzoimidazole-5-carboxylic acid methyl ester

4-Amino-3-fluoro-5-nitro-2-phenylamino-benzoic acid methyl ester (16.70 g, 54.71 mmol), formic acid (250 mL, 6.63 mol) and 20% Pd(OH)₂/C (9.00 g, 16.91 mmol) in ethanol (250 mL) are stirred at 40° C. for two hours under N₂ and then at 95° C. for 16 hours. The reaction mixture is cooled to room temperature and filtered through Celite rinsing with ethyl acetate. The filtrate is concentrated under reduced pressure to give a yellow solid. The solid is triturated with diethyl ether to give 13.47 g (86%) of the desired product as a tan solid: MS APCI (+) m/z 286 (M+1) detected; MS APCI (−) m/z 284 (M−1) detected.

Step C: 7-Fluoro-6-(4-iodo-phenylamino)-3H-benzoimidazole-5-carboxylic acid methyl ester

7-Fluoro-6-phenylamino-3H-benzoimidazole-5-carboxylic acid methyl ester (1.47 g, 4.91 mmol) is suspended in 1:1 THF:MeOH (40 ml) and cooled to −78° C. Solid pTsOH monohydrate (1.5 g, 7.4 mmol) is added followed 5 min later by NIS (1.2 g, 5.2 mmol). After 15 min, the reaction mixture is warmed to 0° C. and then slowly warmed to rt over 16 h. The reaction mixture is quenched by the addition of 10% NaHSO₃. After 30 min, the reaction mixture is poured into a separatory funnel and the layers separated. The aqueous layer is extracted with ethyl acetate. The combined organic extracts are washed with water and brine, dried (Na₂SO₄) and concentrated under reduced pressure. The residue is trituated with methylene chloride to give 1.47 g (69%) pure desired product as a reddish solid: LC/MS ESI (+) m/z 412 (M+1) detected.

Step D: 6-(2-Chloro-4-iodo-phenylamino)-7-fluoro-3H-benzoimidazole-5-carboxylic acid methyl ester

7-Fluoro-6-(4-iodo-phenylamino)-3H-benzoimidazole-5-carboxylic acid methyl ester (1.4 g, 3.5 mmol) is dissolved in DMF (60 ml) and NCS (470 mg, 3.51 mmol) is added. The reaction mixture is stirred 144 h at rt, and then heated to 60° C. After 40 h at 60° C., the reaction mixture is cooled to rt and quenched with 10% NaHSO₃ and diluted with diethyl ether. The layers are separated and the organic layer is washed with water, dried (Na₂SO₄) and concentrated under reduced pressure to give 1.24 g (80%) of desired product as a brown solid: ¹H NMR (400 MHz, DMSO-d₆) δ 8.50 (s, 1H), 7.97 (s, 1H), 7.78 (d, 1H), 7.42 (dd, 1H), 6.1 (bs, 1H), 3.82 (s, 3H).

Step E: 6-(2-Chloro-4-iodo-phenylamino)-7-fluoro-3-methyl-3H-benzoimidazole-5-carboxylic acid methyl ester

6-(2-Chloro-4-iodo-phenylamino)-7-fluoro-3H-benzoimidazole-5-carboxylic acid methyl ester (205 mg, 0.46 mmol) is dissolved in DMF (3 ml) and K₂CO₃ is added (76 mg, 0.55 mmol) followed by MeI (36 μl,0.58 mmol). After 2 h, the reaction mixture is concentrated under reduced pressure to near dryness. The residue is dissolved in ethyl acetate and washed with saturated NaHCO₃ and brine, dried (Na₂SO₄) and concentrated under reduced pressure. Purification by FCC eluted with 9:1 methylene chloride:MeCN gives 35 mg (17%) desired product: ¹H NMR (400 MHz, MeOH-d₄) δ 8.38 (s, 1H), 8.17 (s, 1H), 7.67 (d, 1H), 7.39 (dd, 1H), 6.40 (dd, 1H), 3.98 (s, 3H), 3.93 (s, 3H); ¹⁹F NMR (376 MHz, MeOH-d₄) δ 133.8 (s).

Step F: 6-(2-Chloro-4-iodo-phenylamino)-7-fluoro-3-methyl-3H-benzoimidazole-5-carboxylic acid cyclopropylmethoxy-amide

6-(2-Chloro-4-iodo-phenylamino)-7-fluoro-3-methyl-3H-benzoimidazole-5-carboxylic acid methyl ester is carried forward as described in Example 1 to give 6-(2-chloro-4-iodo-phenylamino)-7-fluoro-3-methyl-3H-benzoimidazole-5-carboxylic acid cyclopropylmethoxy-amide (11b): ¹H NMR (400 MHz, acetone-d₆) δ 8.24 (s, 1H), 7.79 (s, 1H), 7.68 (d, 1H), 7.45 (dd, 1H), 6.41 (dd, 1H), 4.01 (s, 3H), 3.75 (m, 2H), 1.09 (m, 1H), 0.51 (m, 2H), 0.23 (m, 2H).

### Example 3

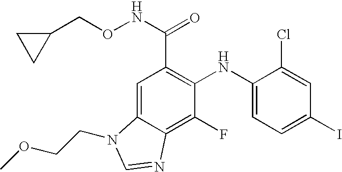

6-(2-Chloro-4-iodo-phenylamino)-7-fluoro-3-(2-methoxy-ethyl)-3H-benzoimidazole-5-carboxylic acid cyclopropylmethoxy-amide (11c)

6-(2-Chloro-4-iodo-phenylamino)-7-fluoro-3-(2-methoxy-ethyl)-3H-benzoimidazole-5-carboxylic acid cyclopropylmethoxy-amide (11c) is prepared from 6(2-chloro-4-iodo-phenylamino)-7-fluoro-3H-benzoimidazole-5-carboxylic acid methyl ester and 1-bromo-2-methoxy-ethane and carried forward as previously described: ¹H NMR (400 MHz, MeOH-d₄) δ 8.32 (s, 1H), 7.72 (s, 1H), 7.63 (m, 1H), 7.33 (dd, 1H), 6.27 (m, 1H), 4.50 (t, 2H), 3.77 (t, 2H), 3.61 (dd, 2H), 3.37 (s, 3H), 1.06 (m, 1H), 0.51 (m, 2H), 0.22 (m, 2H); ¹⁹F NMR (376 MHz, MeOH-d₄) 6-134.91 (s).

### Example 4

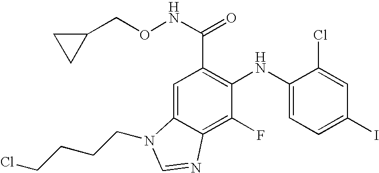

3-(4-Chloro-butyl)-6-(2-chloro-4-iodo-phenylamino)-7-fluoro-3H-benzoimidazole-5-carboxylic acid cyclopropylmethoxy-amide (11d)

3-(4-Chloro-butyl)-6-(2-chloro-4-iodo-phenylamino)-7-fluoro-3H-benzomidazole-5-carboxylic acid cyclopropylmethoxy-amide (11d) is prepared from 6-(2-chloro-4-iodo-phenylamino)-7-fluoro-3H-benzoimidazole-5-carboxylic acid methyl ester and 1-bromo-4-chloro-butane and carried forward as previously described: MS APCI (−) m/z 589, 591, 593 (M-, Cl pattern) detected.

### Example 5

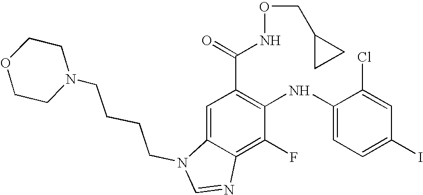

6-(2-Chloro-4-iodo-phenlyamino)-7-fluoro-3-(4-morpholin-4-yl-butyl)-3H-benzoimidazole-5-carboxylic acid cyclopropylmethoxy-amide (11e) 3-(4-Chloro-butyl)-6-(2-chloro-4-iodo-phenylamino)-7-fluoro-3H-benzoimidazole-5-carboxylic acid cyclopropylmethoxy-amide (11d) (45 mg, 0.076 mmol) is dissolved in DMF (0.5 ml) in a pressure tube reactor and NaI (19 mg, 0.12 mmol) is added followed by morpholine (22 μl, 0.25 mmol). The reaction mixture is purged with nitrogen, sealed and heated to 65° C. with stirring for 16 h. The reaction mixture is concentrated under reduced pressure and the residue diluted with ethyl acetate. The organics are washed with water and brine, dried (Na₂SO₄) and concentrated under reduced pressure. Purification by FCC eluted with 95:5 CH₃CN:MeOH gives 36 mg (66%) desired product (11e) as a solid: MS APCI (−) m/z 640, 642 (M-, Cl pattern) detected; ¹H NMR (400 MHz, MeOH-d₄) δ 8.37 (s, 1H), 7.71 (s, 1H), 7.63 (m, 1H), 7.33 (dd, 1H), 6.27 (m, 1H), 4.38 (t, 2H), 3.65 (m, 6H), 2.41 (m, 6H), 1.96 (m, 2H), 1.56 (m, 2H), 1.05 (m, 1H), 0.50 (m, 2H), 0.22 (m, 2H).

### Example 6

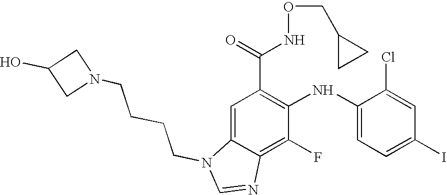

6-(2-Chloro-4-iodo-phenylamino)-7-fluoro-3-[4-(3-hydroxy-azetidin-1-yl)-butyl]-3H-benzoimidazole-5-carboxylic acid cyclopropylmethoxy-amide (11f)

6-(2-Chloro-4-iodo-phenylamino)-7-fluoro-3-[4-(3-hydroxy-azetidin-1-yl)-butyl]-3H-benzoimidazole-5-carboxylic acid cyclopropylmethoxy-amide (11f) is prepared as described above using azetidin-3-ol tosylate and potassium carbonate: MS APCI (−) m/z 626, 628 (M-, Cl pattern) detected; ¹H NMR (400 MHz, MeOH-d₄) δ 8.34 (s, 1H), 7.72 (s, 1H), 7.63 (m, 1H), 7.34 (dd, 1H), 6.27 (m, 1H), 4.34 (m, 3H), 3.61 (m, 3H), 3.38 (m, 2H), 2.86 (m, 2H), 2.54 (m, 2H), 1.95 (m, 2H), 1.41 (m, 1H), 1.06 (m, 1H), 0.51 (m, 2H), 0.22 (m, 2H); ¹⁹F NMR (376 MHz, MeOH-d₄) 6-133.38 (s).

### Example 7

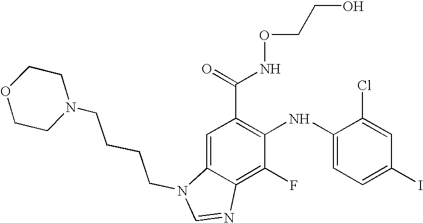

6-(2-Chloro-4-iodo-phenylamino)-7-fluoro-3-(4-morpholin-4-yl-butyl)-3H-benzoimidazole-5-carboxylic acid (2-hydroxy-ethoxy)-amide (11 g)

Step A: 3-(4-Chloro-butyl)-6-(2-chloro-4-iodo-phenylamino)-7-fluoro-3H-benzoimidazole-5-carboxylic acid (2-vinyloxy-ethoxy)-amide

3-(4-Chloro-butyl)-6-(2-chloro-4-iodo-phenylamino)-7-fluoro-3H-benzoimidazole-5-carboxylic acid (70 mg, 0.134 mmol) is suspended in DMF (1 ml) under nitrogen and triethyl amine (44 μl, 0.32 mmol) is added followed by HOBT (25 mg, 0.16 mmol). After 5 min, O-(2-vinyloxy-ethyl)-hydroxylamine (WO 0206213) (17 mg, 0.16 mmol) is added followed by EDCI (31 mg, 0.16 mmol). After 16 h, the reaction mixture is diluted with 1:1 ethyl acetate:THF. The organics are washed with saturated NaHCO₃, saturated NH₄Cl, and brine, and dried (Na₂SO₄) and concentrated under reduced pressure. Purification by trituation with methylene chloride gives 80 mg (98%) desired product: MS APCI (−) m/z 605, 607, 609 (M-, Cl pattern) detected.

Step B: 6-(2-Chloro-4-iodo-phenylamino)-7fluoro-3-(4-morpholin-4-yl-butyl)-3H-benzoimidazole-5-carboxylic acid (2-vinyloxy-ethoxy)-amide

6-(2-Chloro-4-iodo-phenylamino)-7-fluoro-3-(4-morpholin-4-yl-butyl)-3H-benzoimidazole-5-carboxylic acid (2-vinyloxy-ethoxy)-amide is prepared from 3-(4-chloro-butyl)-6-(2-chloro-4-iodo-phenylamino)-7-fluoro-3H-benzoimidazol-5-carboxylic acid (2-vinyloxy-ethoxy)-amide as described previously: MS APCI (−) m/z 656, 658 (M-, Cl pattern).

Step C: 6-(2-Chloro-4-iodo-phenylamino)-7-fluoro-3-(4-morpholin-4-yl-butyl)-3H-benzoimidazole-5-carboxylic acid (2-hydroxy-ethoxy)-amide

6-(2-Chloro-4-iodo-phenylamino)-7-fluoro-3-(4-morpholin-4-yl-butyl)-3H-benzoimidazole-5-carboxylic acid (2-vinyloxy-ethoxy)-amide (24 mg, 0.036 mmols) is suspended in THF (1 ml) and 1.0 N HCl solution (0.18 ml, 0.182 mmols) is added. After 16 h, the reaction mixture is diluted with ethyl acetate and neutralized with saturated NaHCO₃ solution. The organic layer is washed with brine, dried over MgSO₄ and concentrated under reduced pressure. The crude reaction mixture is purified by FCC eluted with 10% MeOH:DCM to give 12 mg (52%) pure desired product (11g) as a white solid: MS APCI (−) m/z 630, 632 (M-, Cl pattern) detected; ¹H NMR (400 MHz, MeOH-d₄) δ 8.39 (s, 1H), 7.74 (s, 1H), 7.63 (m, 1H), 7.33 (dd, 1H), 6.26 (m, 1H), 4.38 (t, 2H), 3.92 (t, 2H), 3.66 (m, 6H), 2.41 (m, 6H), 1.97 (m, 2H), 1.56 (m, 2H); ¹⁹F NMR (376 MHz, MeOH-d₄) 6-135.94 (s).

### Example 8

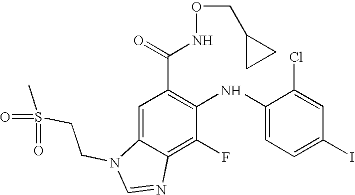

6-(2-Chloro-4-iodo-phenylamino)-7-fluoro-3-(2-methanesulfonyl-ethyl)-3H-benzoimidazole-5-carboxylic acid cyclopropylmethoxy-amide (11h)

Step A: 6-(2-Chloro-4-iodo-phenylamino)-7-fluoro-3-(2-methanesulfonyl-ethyl)-3H-benzoimidazole-5-carboxylic acid methyl ester

6-(2-Chloro-4-iodo-phenylamino)-7-fluoro-3H-benzoimidazole-5-carboxylic acid methyl ester (220 mg, 0.494 mmol) is dissolved in 1:1 THF:DMF (2 ml) under nitrogen and K₂CO₃ (69 mg, 0.499 mmol) is added followed by methyl vinyl sulfone (51 μl, 0.592 mmol). After 16 h, the reaction mixture is concentrated under reduced pressure and the residue is dissolved in ethyl acetate. The organics are washed with saturated NaHCO₃, and brine, and dried (Na₂SO₄) and concentrated under reduced pressure. Purification by FCC eluted with 1:1 methylene chloride:MeCN gives 122 mg (45%) desired product as an off-white solid.

Step B: 6-(2-Chloro-4-iodo-phenylamino)-7-fluoro-3-(2-methanesulfonyl-ethyl)-3H-benzoimidazole-5-carboxylic acid cyclopropylmethoxy-amide

Hydroylsis and coupling as previously described gives desired product (11h): MS APCI (−) m/z 605, 607 (M-, Cl pattern) detected; ¹H NMR (400 MHz, acetone-d₆) δ 10.95 (bs, 1H), 8.37 (s, 1H), 8.21 (bs, 1H), 7.92 (s, 1H), 7.70 (d, 1H), 7.46 (dd, 1H), 6.44 (m, 1H), 4.93 (t, 2H), 3.85 (t, 2H), 3.75 (dd, 2H), 2.98 (s, 3H) 1.09 (m, 1H), 0.44 (m, 2H), 0.24 (m, 2H); ¹⁹F NMR (376 MHz, acetone-d₆) 6-132.31 (s).

### Example 9

The following compounds are prepared similarly using the appropriate Michael acceptor and hydroxylamine.

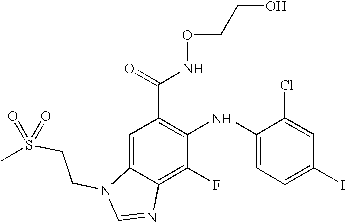

6-(2-Chloro-4-iodo-phenylamino)-7-fluoro-3-(2-methanesulfonyl-ethyl)-3H-benzoimidazole-5-carboxylic acid (2-hydroxy-ethoxy)-amide (11i): MS APCI (−) m/z 595, 597 (M-, Cl pattern) detected; ¹H NMR (400 MHz, MeOH-d₄) δ 8.39 (s, 1H), 7.78 (s, 1H), 7.64 (d, 1H), 7.34 (dd, 1H), 6.28 (m, 1H), 4.87 (t, 2H), 3.93 (m, 2H), 3.79 (t, 2H), 3.67 (m, 2H) 2.98 (s, 3H); ¹⁹F NMR (376 MHz, MeOH-d₄) 6-134.00 (s).

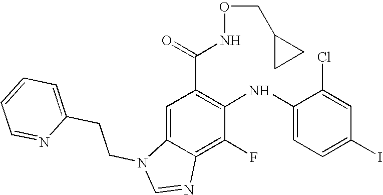

6-(2-Chloro-4-iodo-phenylamino)-7-fluoro-3-(2-pyridin-2-yl-ethyl)-3H-benzoimidazole-5-carboxylic acid cyclopropylmethoxy-amide (11j): MS APCI (+) m/z

606, 608 (M+, Cl pattern) detected; MS APCI (−) m/z 604, 606 (M-, Cl pattern) detected; ¹H NMR (400 MHz, MeOH-d₄) δ 8.47 (d, 1H), 8.13 (s, 1H), 7.65 (dt, 1H), 7.62 (m, 2H), 7.35 (dd, 1H), 7.26 (dd, 2H), 7.20 (d, 1H), 6.25 (dd, 1H), 4.75 (t, 2H), 3.62 (d, 2H), 3.39 (t, 2H), 1.09 (m, 1H), 0.51 (m, 2H), 0.25 (m, 2H); ¹⁹F NMR (376 MHz, MeOH-d₄) δ-134.62 (s).

The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A compound of the formula 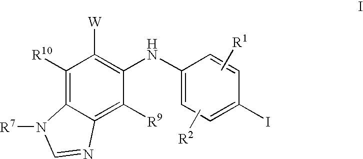
and pharmaceutically accepted salts, prodrugs and solvates thereof, wherein:
R¹, R², R⁹ and R¹⁰ are independently selected from hydrogen, halogen, cyano, nitro, trifluoromethyl, difluoromethoxy, trifluoromethoxy, azido, —OR³, —C(O)R³, —C(O)OR³, NR⁴C(O)OR⁶, —OC(O)R³, —NR⁴SO²R⁶, —SO₂NR³R⁴, —NR⁴C(O)R³, —C(O)NR³R⁴, —NR⁵C(O)NR³R⁴, —NR⁵C(NCN)NR³R⁴, —NR³R⁴, and
C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR⁴R⁵)ₘ-aryl, aryl, arylalkyl, heteroaryl, heteroarylalkyl, heterocyclyl, heterocyclylalkyl, —O(CR⁴R⁵)ₘ-aryl, —NR⁴(CR⁴R⁵)ₘ-aryl, —O(CR⁴R⁵)ₘ-heteroaryl, —NR⁴(CR⁴R⁵)ₘ-heteroaryl, —O(CR⁴R⁵)ₘ-heterocyclyl and —NR⁴(CR⁴R⁵)ₘ-heterocyclyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —NR⁴C(O)OR⁶, —NR⁴C(O)R³, —C(O)NR³R⁴, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl;
R³ is selected from hydrogen, trifluoromethyl, and
C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR′SO₂R″″—SO₂NR′R″, —C(O)R′, —C(O)OR′, —OC(O)R′, —NR′C(O)OR″″, NR′C(O)R″, —C(O)NR′R″, —SR″″, —S(O)R″″, —SO₂R′, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl;
R′, R″ and R′″ independently are selected from hydrogen, lower alkyl, lower alkenyl, aryl and arylalkyl;
R″″ is selected from lower alkyl, lower alkenyl, aryl and arylalkyl; or
Any two of R′, R″, R′″ or R″″ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl; or
R³ and R⁴ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R″, —NR′C(O)OR″″, —NR′C(O)R″, —C(O)NR′R″, —SO₂R″″, —NR′R″, —NR′C(O)NR″R′″, NR′C(NCN)NR″R′″, —OR′, aryl, heteroaryl, arylalkyl, heteroarylalkyl, heterocyclyl, and heterocyclylalkyl; or
R⁴ and R⁵ independently represent hydrogen or C₁-C₆ alkyl; or
R⁴ and R⁵ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, —NR′SO₂R″″, —SO₂NR′R″″, —C(O)R″″, —C(O)OR′, —OC(O)R′, —NR′C(O)OR″, —NR′C(O)R″, —C(O)NR′R″, —SO₂R″″, —NR′R″, —NR′C(O)NR″R′″, NR′C.(NCN)NR″R′″, —OR′, aryl, heteroaryl, arylalkyl, heteroarylalkyl, heterocyclyl, and heterocyclylalkyl;
R⁶ is selected from trifluoromethyl; and
C₁-C₁₀alkyl, C₃-C₁₀ cycloalkyl, aryl, arylalkyl, heteroaryl, heteroarylalkyl, heterocyclyl, and heterocyclylalkyl, where each alkyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR′C(O)OR″″, NR′C(O)R″, —C(O)NR′R″, —SO₂R″″, —NR′R′, —NR′C(O)NR″R′″, —NR′C(NCN)NR″R′″, —OR′, aryl, heteroaryl, arylalkyl, heteroarylalkyl, heterocyclyl, and heterocyclylalkyl;
R⁷ is selected from hydrogen; and
C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —NR⁴C(O)OR⁶, —NR⁴C(O)R³, —C(O)NR³R⁴, —SO₂R⁶, —NR³R⁴, —NR⁵C(O)NR³R¹⁴, —NR⁵C(NCN)NR³, R⁴, —OR³, aryl, heteroaryl, arylalkyl, heteroarylalkyl, heterocyclyl, and heterocyclylalkyl;
W is selected from heteroaryl, heterocyclyl, —C(O)OR³, —C(O)NR³R⁴, —C(O)NR⁴OR³, —C(O)R⁴OR³, —C(O)(C₃-C₁₀ cycloalkyl), —C(O)(C₁-C₁₀ alkyl), —C(O)(aryl), —C(O)(heteroaryl) and —C(O)(heterocyclyl), each of which is optionally substituted with 1-5 groups independently selected from
—NR³R⁴, —OR³, —R², and
C₁-C₁₀ alkyl, C₂-C₁₀ alkenyl, and C₂-C₁₀ alkynyl, each of which is optionally substituted with 1 or 2 groups independently selected from —NR³R⁴ and —OR³;
m is 0, 1, 2, 3, 4 or 5; and
j is 1 or 2.

2. A compound of the formula 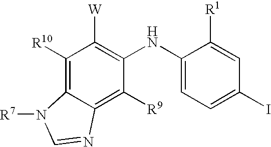
and pharmaceutically accepted salts, prodrugs and solvates thereof, wherein:
R¹, R⁹ and R¹⁰ are independently selected from hydrogen, halogen, cyano, nitro, trifluoromethyl, difluoromethoxy, trifluoromethoxy, azido, —OR³, —C(O)R³, —C(O)OR³, NR⁴C(O)OR⁶, —OC(O)R³, —NR⁴SO₂R⁶, —SO₂NR³R⁴, —NR⁴C(O)R³, —C(O)NR³R⁴, —NR⁵C(O)NR³R⁴, —NR⁵C(NCN)NR³R, —NR³R⁴, and
C₁-C₁₀ alkyl, C₂-C₁₀ alkenyl, C₂-C₁₀alkynyl, C₃-C₁₀ cycloalkyl, C₃-C₁₀ cycloalkylalkyl, —S(O)ⱼ(C₁-C₆ alkyl), —S(O)ⱼ(CR⁴R⁵)ₘ-aryl, aryl, arylalkyl, heteroaryl, heteroarylalkyl, heterocyclyl, heterocyclylalkyl, —O(CR⁴R⁵)ₘ-aryl, —NR⁴(CR⁴R⁵)ₘ-aryl, —O(CR⁴R⁵)ₘ-heteroaryl, —NR⁴(CR⁴R⁵)ₘ-heteroaryl, —O(CR⁴R⁵)ₘ-heterocyclyl and —NR⁴(CR⁴R⁵)ₘ-heterocyclyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —NR⁴C(O)OR⁶, —NR⁴C(O)R³, —C(O)NR³R⁴, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl;
R³ is selected from hydrogen, trifluoromethyl, and
C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR′C(O)OR″″, —NR′C(O)R″, —C(O)NR′R″, —SR″″, —S(O)R″″, —SO₂R′, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl;
R′, R″ and R′″ independently are selected from hydrogen, lower alkyl, lower alkenyl, aryl and arylalkyl;
R″″ is selected from lower alkyl, lower alkenyl, aryl and arylalkyl; or
Any two of R′, R″, R′″ or R″″ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl; or
R³ and R⁴ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR C(O)OR″″,—NR′C(O)R″, —C(O)NR′R″, —SO₂R″″, —NR′R″, —NR′C(O)NR″R′″, NR′C(NCN)NR″R′″, —OR′, aryl, heteroaryl, arylalkyl, heteroarylalkyl, heterocyclyl, and heterocyclylalkyl; or
R⁴ and R⁵ independently represent hydrogen or C₁-C₆ alkyl; or
R⁴ and R⁵ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR′C(O)OR″″, —NR′C(O)R″, —C(O)NR′R″, —SO₂R″, —NR′R″—NR′C(O)NR″R′″, —NR′C(NCN)NR″R′″, —OR′, aryl, heteroaryl, arylalkyl, heteroarylalkyl, heterocyclyl, and heterocyclylalkyl;
R is selected from trifluoromethyl; and
C₁-C₁₀ alkyl, C₃-C₁₀ cycloalkyl, aryl, arylalkyl, heteroaryl, heteroarylalkyl, heterocyclyl, heterocyclylalkyl, where each alkyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR′(O)OR″″, —NR′C(O)R″, —C(O)NR″R′″, —SO₂R′″, —NR′R′, —NR′C(O)NR″R′″, —NR′C.(NCN)NR″R′″, —OR′, aryl, heteroaryl, arylalkyl, heteroarylalkyl, heterocyclyl, and heterocyclylalkyl;
R⁷ is selected from hydrogen; and
C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, heterocyclylalkyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —NR⁴C(O)OR⁶, —NR⁴C(O)R³, —C(O)NR³R⁴, —SO₂R⁶, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl;
W is selected from heteroaryl, heterocyclyl, —C(O)OR³, —C(O)NR³R⁴, —C(O)NR⁴OR³, —C(O)R⁴OR³, —C(O)(C₃-C₁₀ cycloalkyl), —C(O)(C₁-C₁₀ alkyl), —C(O)(aryl), —C(O)(heteroaryl) and —C(O)(heterocyclyl), each of which is optionally substituted with 1-5 groups independently selected from
—NR³R⁴, —OR³, —R², and
C₁-C₁₀ alkyl, C₂-C₁₀alkenyl, and C₂-C₁₀alkynyl, each of which is optionally substituted with 1 or 2 groups independently selected from —NR³R⁴ and —OR³;
m is 0, 1, 2, 3, 4 or 5; and
j is 1 or 2.

3. A compound of the formula 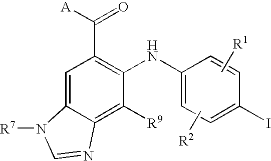
and pharmaceutically accepted salts, prodrugs and solvates thereof, wherein:
R¹, R², and R⁹ are independently selected from hydrogen, halogen, cyano, nitro, trifluoromethyl, difluoromethoxy, trifluoromethoxy, azido, —OR³, —C(O)R³, —C(O)OR³, NR⁴C(O)OR⁶, —OC(O)R³, —NR⁴SO₂R⁶, —SO₂NR³R⁴, —NR⁴C(O)R³, —C(O)NR³R⁴, —NR⁵C(O)NR³R⁴, —NR⁵C(NCN)NR³R⁴, —NR³R⁴, and
C₁-C₁₀ alkyl, C₂-C₁₀alkenyl, C₂-C₁₀alkynyl, C₃-C₁₀ cycloalkyl, C₃-C₁₀ cycloalkylalkyl, —S(O)ⱼ(C₁-C₆ alkyl), —S(O)ⱼ(CR⁴R⁵)ₘ-aryl, aryl, arylalkyl, heteroaryl, heteroarylalkyl, heterocyclyl, heterocyclylalkyl, —O(CR⁴R⁵)ₘ-aryl, —NR⁴(CR⁴R⁵)ₘ-aryl, —O(CR⁴R⁵)ₘ-heteroaryl, —NR⁴(CR⁴R⁵)ₘ-heteroaryl, —O(CR⁴R⁵)ₘ-heterocyclyl and —NR⁴(CR⁴R⁵)ₘ-heterocyclyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —NR⁴C(O)OR⁶, —NR⁴C(O)R³, —C(O)NR³R⁴, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl;
R³ is selected from hydrogen, trifluoromethyl; and
C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, heterocyclylalkyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR′C(O)OR″″, —NR′C(O)R″, —C(O)NR′R″, —SR′, —S(O)R″″, —SO₂R″″, —NR′R″, —NR C(O)NR″R′″, —NR′C(CN)NR′R″, —OR′, aryl, heteroaryl, arylalkyl, heteroarylalkyl, heterocyclyl, and heterocyclylalkyl;
R′, R″ and R′″ independently are selected from hydrogen, lower alkyl, lower alkenyl, aryl and arylalkyl;
R″″ is selected from lower alkyl, lower alkenyl, aryl and arylalkyl; or
Any two of R′, R″, R′″ or R″″ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl; or
R³ and R⁴ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl; or
R⁴ and R⁵ independently represent hydrogen or C₁-C₆ alkyl, or
R⁴ and R⁵ can be taken together with the atom to which they are attached to form a 4 to 10 membered carbocyclic, heteroaryl or heterocyclic ring, each of which is optionally substituted with one to three groups independently selected from halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR′C(O)OR″″, —NR′C(O)R″, —C(O)NR′R″, —SO₂R″″, —NR′R″, —NR′C(O)NR″R′″, NR′C(NCN)NR″R′″, —OR′, aryl, heteroaryl, arylalkyl, heteroarylalkyl, heterocyclyl, and heterocyclylalkyl;
R⁶ is selected from trifluoromethyl; and
C₁-C₁₀ alkyl, C₃-C₁₀ cycloalkyl, aryl, arylalkyl, heteroaryl, heteroarylalkyl, heterocyclyl, heterocyclylalkyl, where each alkyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR′SO₂R″″, —SO₂NR′R″, —C(O)R′, —C(O)OR′, —OC(O)R′, —NR′C(O)OR″″, NR′C(O)R″, —C(O)NR′R″, —SO₂R′″, —NR′R′, —NR′C(O)NR″R′″, NR′C(NCN)NR″R′″, —OR′, aryl, heteroaryl, arylalkyl, heteroarylalkyl, heterocyclyl, and heterocyclylalkyl;
R⁷ is selected from hydrogen; and
C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, heterocyclylalkyl, where each alkyl, alkenyl, alkynyl, cycloalkyl, aryl, heteroaryl and heterocyclyl portion is optionally substituted with one to five groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³,—OC(O)R³, —NR⁴C(O)OR⁶, —NR⁴C(O)R³—C(O)NR³R⁴, —SO₂R⁶, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl;
A is selected from —C(O)OR³ or —C(O)NR⁴OR³;
m is 0, 1, 2, 3, 4 or 5; and
j is 1 or 2.

4. A compound according to claim 3 having the formula

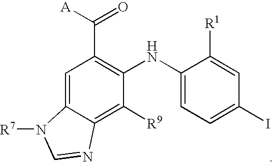

5. A compound according to claim 4 wherein
R⁷ is C₁-C₁₀ alkyl, C₃-C₇ cycloalkyl, C₃-C₇ cycloalkylalkyl, C₃-C₇ heterocycloalkyl or C₃-C₇ heterocycloalkylalkyl, each of which can be optionally substituted with 1-3 groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)R³, —OC(O)R³, —SO₂R³, —NR⁴C(O)OR⁶, —NR⁴C(O)R³, —C(O)NR 3R⁴, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl;
R⁹ is hydrogen or halogen; and
R¹ is lower alkyl or halogen.

6. A compound according to claim 5 wherein R⁹ is fluoro.

7. A compound according to claim 6 wherein R¹ is methyl or chloro.

8. A compound according to claim 5 wherein A is —C(O)NR⁴OR³.

9. A compound according to claim 1 wherein
R⁷ is C₁-C₁₀ alkyl, C₃-C₇ cycloalkyl, C₃-C₇ cycloalkylalkyl, C₃-C₇ heterocycloalkyl or C₃-C₇ heterocycloalkylalkyl, each of which can be optionally substituted with 1-3 groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —SO₂R³, —NR⁴C(O)OR⁶, —NR⁴C(O)R³, —C(O)NR³R⁴, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl;
R⁹ is hydrogen or halogen;
R¹⁰ is hydrogen; and
W is —C(O)OR³ or —C(O)NR⁴OR³.

10. A compound according to claim 9 wherein W is —C(O)NR⁴OR³.

11. A compound according to claim 2 wherein
R⁷ is C₁-C₁₀ alkyl, C₃-C₇ cycloalkyl, C₃-C₇ cycloalkylalkyl, C₃-C₇ heterocycloalkyl or C₃-C₇ heterocycloalkylalkyl, each of which can be optionally substituted with 1-3 groups independently selected from oxo, halogen, cyano, nitro, trifluoromethyl, difluoromethoxy, trifluoromethoxy, azido, —NR⁴SO₂R⁶, —SO₂NR³R⁴, —C(O)R³, —C(O)OR³, —OC(O)R³, —SO₂R⁶, —NR⁴C(O)OR⁶, —NR⁴C(O)R³, —C(O)NR³R⁴, —NR³R⁴, —NR⁵C(O)NR³R⁴, —NR⁵C(NCN)NR³R⁴, —OR³, aryl, heteroaryl, arylalkyl, heteroarylalkyl, heterocyclyl, and heterocyclylalkyl;
R⁹ is hydrogen or halogen;
R¹⁰ is hydrogen; and
W is —C(O)OR³ or —C(O)NR⁴OR³.

12. A compound according to claim 11 wherein W is —C(O)NR⁴OR³.

13. A compound according to claim 1 which is selected from
7-Fluoro-6-(4-iodo-2-methyl-phenylamino)-3-methyl-3H-benzoimidazole-5-carboxylic acid cyclopropylmethoxy-amide;
6-(2-Chloro-4-iodo-phenylamino)-7-fluoro-3-methyl-3H-benzoimidazole-5-carboxylic acid cyclopropylmethoxy-amide;
6-(2-Chloro-4-iodo-phenylamino)-7-fluoro-3-(2-methoxy-ethyl)-3H-benzoimidazole-5-carboxylic acid cyclopropylmethoxy-amide;
3-(4-Chloro-butyl)-6-(2-chloro-4-iodo-phenylamino)-7-fluoro-3H-benzoimidazole-5-carboxylic acid cyclopropylmethoxy-amide;
6-(2-Chloro-4-iodo-phenylamino)-7-fluoro-3-(4-morpholin-4-yl-butyl)-3H-benzoimidazole-5-carboxylic acid cyclopropylmethoxy-amide;
6-(2-Chloro-4-iodo-phenylamino)-7-fluoro-3-[4-(3-hydroxy-azetidin-1-yl)-butyl]-3H-benzoimidazole-5-carboxylic acid cyclopropylmethoxy-amide;
6-(2-Chloro-4-iodo-phenylamino)-7-fluoro-3-(4-morpholin-4-yl-butyl)-3H-benzoimidazole-5-carboxylic acid (2-hydroxy-ethoxy)-amide;
6-(2-Chloro-4-iodo-phenylamino)-7-fluoro-3-(2-methanesulfonyl-ethyl)-3H-benzoimidazole-5-carboxylic acid cyclopropylmethoxy-amide;
6-(2-Chloro-4-iodo-phenylamino)-7-fluoro-3-(2-methanesulfonyl-ethyl)-3H-benzoimidazole-5-carboxylic acid (2-hydroxy-ethoxy)-amide; and
6-(2-Chloro-4-iodo-phenylamino)-7-fluoro-3-(2-pyridin-2-yl-ethyl)-3H-benzoimidazole-5-carboxylic acid cyclopropylmethoxy-amide.

14. A composition comprising a compound of claim 1 and a pharmaceutically acceptable carrier.

15. A composition comprising a compound of claim 13 and a pharmaceutically acceptable carrier.

16. A method of inhibiting MEK activity in a mammal comprising administrating an effective amount of a compound of claim 1 to the mammal.

17. A method for treatment of cancer in a mammal comprising administrating an effective amount of a compound of claim 1 to the mammal.
